(19) United States
(12) Patent Application Publication
(10) Pub. No.: US 2011/0183314 A1
(43) Pub. Date: Jul. 28, 2011

(54) BACTERIOPHAGE-BASED MICROORGANISM DIAGNOSTIC ASSAY USING SPEED OR ACCELERATION OF BACTERIOPHAGE REPRODUCTION

(75) Inventor: Jonathan D. Smith, Boulder, CO (US)

(73) Assignee: MicroPhage Incorporated, Longmont, CO (US)

(21) Appl. No.: 13/014,631

(22) Filed: Jan. 26, 2011

Related U.S. Application Data
(60) Provisional application No. 61/298,438, filed on Jan. 26, 2010.

(51) Int. Cl. C12Q 1/70

(52) U.S. Cl. 435/5

(57) ABSTRACT

A method of determining the presence or absence of a target microorganism in a sample to be tested, the method comprising: combining with the sample an amount of bacteriophage capable of infecting the target microorganism to create a bacteriophage-exposed sample; and measuring the time rate of change of the amount of said bacteriophage or the change in the rate of change of the amount of said bacteriophage as an indication of the presence or absence of the target microorganism as a function of time.

### CROSS-REFERENCE TO RELATED APPLICATIONS

This application claims the benefit of U.S. Provisional Application No. 61/298,438 filed on Jan. 26, 2010, titled “Bacteriophage-Based Microorganism Diagnostic Assay Using Speed Or Acceleration Of Bacteriophage Reproduction,” the entire disclosure of which is hereby incorporated by reference.

### FIELD OF THE INVENTION

The invention relates generally to the field of identification of microscopic living organisms and more particularly to the identification of microorganisms using bacteriophage.

### BACKGROUND OF THE INVENTION

Currently, bacteria that may be causing an infection or other health problem are identified by bacteria culture methods. Generally, it takes a day or several days to grow sufficient bacteria to enable the detection and identification of the bacteria. By that time, the person or persons infected by the bacteria may be very sick or even dead. Thus, there is a need for more rapid detection and identification of bacteria. Further, when bacterial infection is suspected, a physician will often prescribe a broad spectrum antibiotic. This has led to the development of antibiotic-resistant bacteria, which has further enhanced the need for more rapid identification of bacteria.

Bacteriophage are ubiquitous viruses that infect bacteria. Bacteriophage-based methods have been suggested as a method to accelerate bacterial identification. Bacteriophage are viruses that have evolved in nature to use bacteria as a means of replicating themselves. A bacteriophage (or phage) does this by attaching itself to a bacterium and injecting its genetic material into that bacterium, inducing it to replicate the phage from tens to thousands of times. Some bacteriophage, called “lytic bacteriophage,” rupture the host bacterium, releasing the progeny phage into the environment to seek out other bacteria. Thus, because of the sheer number of the bacteriophage after amplification, in principle it should be easier to detect the bacteriophage than to detect the bacteria. If, in addition the bacteriophage is specific to the bacteria, that is, if the bacteriophage amplification of a particular bacteriophage only occurs for specific bacteria, then the presence of amplified bacteria is then also an indication of the presence of the bacteria to which it is specific. Further, since the total incubation time for infection of a bacterium by parent phage, phage multiplication (amplification) in the bacterium to produce progeny phage, and release of the progeny phage after lysis can take as little as an hour after the bacteriophage find the bacteria depending on the phage, the bacterium, and the environmental conditions, in principle, bacteriophage amplification can result in much faster detection and identification of bacteria. See, for example, U.S. Pat. No. 5,985,596 issued Nov. 16, 1999 and No. 6,461,833 B1 issued Oct. 8, both to Stuart Mark Wilson; and Angelo J. Madonna, Sheila VanCuyk and Kent J. Voorhees, “Detection Of Esherichia Coli Using Immunomagnetic Separation And Bacteriophage Amplification Coupled With Matrix-Assisted Laser Desorption/Ionization Time-Of-Flight Mass Spectrometry”, Wiley InterScience, DOI:10.1002/rem.900, 24 Dec. 2002, which references are hereby incorporated by reference to the same extent as though fully disclosed herein. In summary, because bacteriophage are obligate bacterial parasites, their growth is fully dependent upon the presence of a suitable viable bacterial host. Bacteriophage amplification thus can be used as a surrogate marker for the identification and characterization of bacteria in a sample of interest, providing information that is of value in food, clinical, and environmental testing.

Bacteriophage amplification assays that depend upon amplification above a threshold level have been described: detection of bacteriophage at a concentration over a predetermined threshold is taken to indicate the presence of a suitable viable host in the sample.

In each of the methods of the above references, samples potentially containing target bacteria are incubated with bacteriophage, as specific as possible for those bacteria. In the presence of the bacteria, the bacteriophage infect the bacteria and replicate in the bacteria, resulting in the production of a measurable signal indicating the presence of the target bacteria. Some methods utilize the detection of progeny phage released from infected target bacteria as a means of detection and identification. In this case, progeny phage are not produced if the parent phage do not successfully infect the target bacteria. The degree to which the phage will infect the bacteria if the phage and bacteria are in the same sample is called “the infectious sensitivity of the phage.” Still other methods rely on the detection of phage replication products rather than whole progeny phage. For example, luciferase reporter bacteriophage produce luciferase when they successfully infect target bacteria. The luciferase then produces light that, if detected, indicates the presence of target bacteria in the sample. The promise of these methods has lead to much research on bacteriophage-based identification of microorganisms. However, as of this writing, the only commercially successful method of bacteriophage-based identification is a process in which the concentration of the bacteria is enhanced by a blood culturing process before or while the bacteriophage-based bacteria identification is performed.

In any method based on phage amplification, it is necessary to separate the signal that arises from the parent bacteriophage from the signal from the progeny bacteriophage. U.S. Pat. No. 5,498,525 issued Mar. 12, 1996 to Rees et al. solves this problem by destroying, removing, neutralizing, or inactivating the parent bacteriophage; and U.S. Pat. No. 7,166,425 issued Jan. 23, 2007 to Madonna et al. solves this problem by using a quantity of parent bacteriophage that is below the detection limit of the detection technology. However, to be sure that a lower level of bacteria are detected, the quantity of bacteriophage is kept as high as possible while still being under the detection limit.

To reliably detect a signal, the threshold must be significantly larger than the variability in initial bacteriophage concentration across sample runs. This variation can be attributed to many factors, including operator or manufacturing variability, dilution by sample, loss of activity over the test shelf life, or inhibition or neutralization by sample interferents.

Clearly, it would be highly desirable if a bacteriophage process could be provided that had increased selectivity, increased infectious sensitivity, and/or increased test sensitivity and still retained the fast detection of bacteria that is the promise of bacteriophage amplification methods, the potential of which has been driving research in this field.

### BRIEF SUMMARY OF THE INVENTION

The invention solves the above problems, as well as other problems of the prior art, by employing the change in bacteriophage concentration over time, the curvature of a plot of bacteriophage concentration over time, or the change in the rate of change of bacteriophage concentration over time as the indicator of the presence of a specific bacterial host within the sample.

The invention provides a method of determining the presence or absence of a target microorganism in a sample to be tested, said method comprising: (a) combining with said sample an amount of bacteriophage capable of infecting said target microorganism to create a bacteriophage-exposed sample; (b) providing conditions to said bacteriophage-exposed sample sufficient to allow said bacteriophage to multiply in said target microorganism; and (c) assaying said bacteriophage-exposed sample to detect the time rate of change of a bacteriophage marker to determine the presence or absence of said target microorganism. Preferably, said microorganism is a bacterium, and said assaying comprises detecting said bacteriophage marker as an indication of the presence of said target bacterium in said sample. Preferably, said rate of change is the first time derivative or curvature of said bacteriophage marker. Preferably, said rate of change is the second time derivative of said bacteriophage marker. Preferably, said assaying comprises applying an algorithm that detects the slope of said marker. Preferably, the initial amount of said bacteriophage comprises a bacteriophage concentration of between 1×10³ pfu/mL and 1×10⁷ pfu/mL.

The invention also provides a method of determining the presence or absence of a target microorganism in a sample to be tested, said method comprising: (a) combining with said sample an amount of bacteriophage capable of infecting said target microorganism to create a bacteriophage-exposed sample; (b) providing conditions to said bacteriophage-exposed sample sufficient to allow said bacteriophage to multiply in said target microorganism; and (c) assaying said bacteriophage-exposed sample to detect the time rate of change of the time rate of change in a bacteriophage marker to determine the presence or absence of said target microorganism. Preferably, said microorganism is a bacterium and said assaying comprises detecting said bacteriophage marker as an indication of the presence of said target bacterium in said sample. Preferably, said assaying comprises applying an algorithm that detects the change in slope of said marker as a function of time. Preferably, the initial amount of said bacteriophage comprises a bacteriophage concentration of between 1×10³ pfu/mL and 1×10⁷ pfu/mL.

The invention solves the problem of the noisy bacteriophage marker signal while at the same time increasing the speed of bacterial identification. Numerous other features, objects, and advantages of the invention will become apparent from the following description when read in conjunction with the accompanying drawings.

### BRIEF DESCRIPTION OF THE DRAWING

FIG. 1 is a graph of bacteriophage concentration versus time for three different runs having different starting conditions; and

FIG. 2 is a graph of the first derivative of the three plot traces of FIG. 1.

### DETAILED DESCRIPTION OF THE INVENTION

In this disclosure, the terms “bacteriophage” and “phage” include bacteriophage, phage, mycobacteriophage (such as for TB and paraTB), mycophage (such as for fungi), mycoplasma phage or mycoplasmal phage, and any other term that refers to a virus that can invade living bacteria, fungi, mycoplasmas, protozoa, yeasts, and other microscopic living organisms and uses them to replicate itself. Here, “microscopic” means that the largest dimension is one millimeter or less. Bacteriophage are viruses that have evolved in nature to use bacteria as a means of replicating themselves. A phage does this by attaching itself to a bacterium and injecting its DNA (or RNA) into that bacterium, and inducing it to replicate the phage hundreds or even thousands of times. This is referred to as “phage amplification.”

Whether the bacteriophage has infected the bacteria is determined by an assay that can identify the change in concentration of a bacteriophage or bacterial marker or the change in the rate of change of a bacteriophage or bacterial marker. In this disclosure, a bacteriophage marker is any biological or organic element that can be associated with the presence of a bacteriophage. Without limitation, this may be the bacteriophage itself, a lipid incorporated into the phage structure, a protein associated with the bacteriophage, RNA or DNA associated with the bacteriophage, or any portion of any of the foregoing. In this disclosure, a bacterial marker is any biological or organic element that is released when a bacterium is lysed by a bacteriophage, including cell wall components, bacterial nucleic acids, proteins, enzymes, small molecules, or any portion of the foregoing. Preferably, the assay not only can identify the bacteriophage marker but also the quantity or concentration of the bacteriophage or bacterial marker and the change in the marker. In this disclosure, determining the quantity of a microorganism is equivalent to determining the concentration of the microorganism, since if you have one, you have the other, since the volume of the sample is nearly always known, and, if not known, can be determined. Determining the quantity or concentration of something can mean determining the number, the number per unit volume, determining a range wherein the number or number per unit volume lies, or determining that the number or concentration is below or above a certain critical threshold. Generally, in this art, the amount of a microorganism is given as a factor of ten, for example, 2.3×10⁷ bacteriophage per milliliter (ml).

Some bacteriophage, called lytic bacteriophage, rupture the host bacterium, releasing the progeny phage into the environment to seek out other bacteria. The total reaction time for phage infection of a bacterium, phage multiplication, or amplification in the bacterium, through lysing of the bacterium takes anywhere from tens of minutes to hours, depending on the phage and bacterium in question and the environmental conditions. Once the bacterium is lysed, progeny phage are released into the environment along with all of the contents of the bacteria. The progeny phage will infect other bacteria that are present and repeat the cycle to create more phage and more bacterial debris. In this manner, the number of phage will increase exponentially until there are essentially no more bacteria to infect. The concept underlying the art of using bacteriophage to detect bacteria is that the huge numbers of phage that are created during phage amplification can be detected more easily than the much smaller number of bacteria; thus, phage amplification can be used to detect the presence of bacteria.

A fundamental principle that allows particular bacteria to be detected and identified via bacteriophage amplification followed by an assay of a bacteriophage marker is that a particular bacteriophage will, in principal, infect only a particular bacterium. That is, the bacteriophage is specific to the bacteria. Thus, if a particular bacteriophage that is specific to particular bacteria is introduced into a sample, and later the bacteriophage has been found to have multiplied, the bacteria to which the bacteriophage is specific must have been present in the sample. In this way, the prior art teaches that bacteriophage amplification can be used to identify specific bacteria present in a sample. However, the bacteriophage is rarely, if ever, 100% specific to a bacterium. In nature, bacteriophage tend to generally be 80% or less specific. This creates problems in bacterium detection and identification, and can be an additional factor that adds noise to the signal.

However, as indicated above, bacteriophage-based assays are inherently noisy. The present invention teaches a method of increasing the sensitivity and reliability of bacteriophage-based assays by using the change in bacteriophage concentration over time, i.e., the first derivative of the bacteriophage concentration or curvature, or a change in the rate of change in bacteriophage concentration over time, i.e., the second derivative of the bacteriophage concentration, as the signal that indicates bacteriophage growth, and thus the presence of a bacterial host in the sample, or more specifically, the presence of the bacteria to which the bacteriophage is specific.

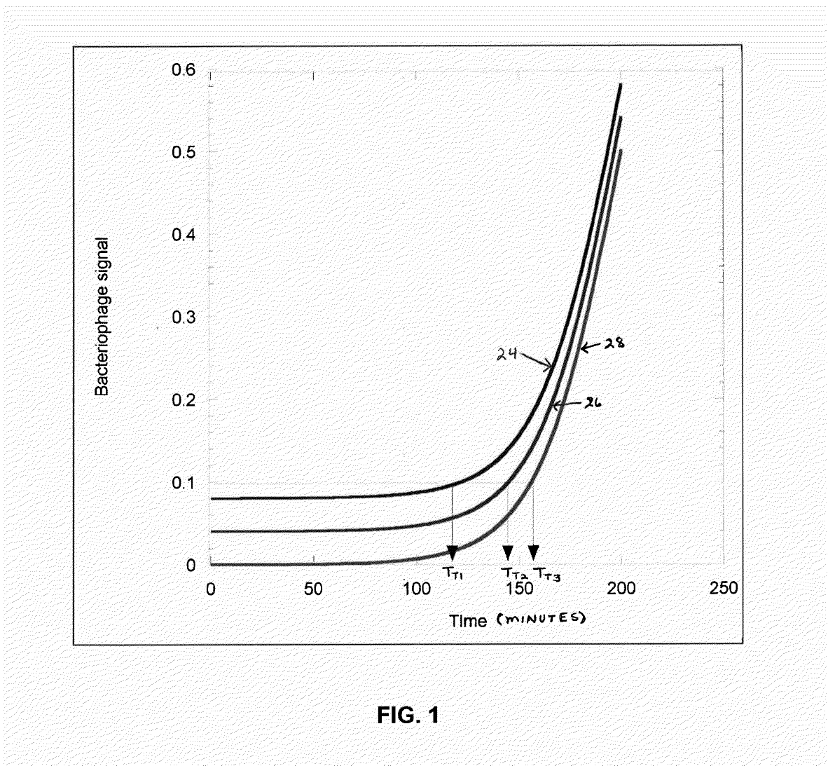

FIG. 1 illustrates this principle. A plot of bacteriophage signal versus time for three different samples having initially different starting conditions, for example, and different concentrations of bacteriophage, is shown. The bacteriophage signal may be any measure of bacteriophage number or concentration. Generally, in this art, bacteriophage concentration is given in pfu/mL. For example, the initial concentration of one sample may be 1×10⁶ pfu/mL; the initial concentration of another sample may be 3×10⁶ pfu/mL; and the initial concentration of a third sample may be 7×10⁶ pfu/mL. The range of bacteriophage initial concentration is preferably between 1×10³ pfu/mL and 1×10⁷ pfu/mL. More preferably, the initial amount of the bacteriophage is between 1×10⁵ pfu/mL and 7×10⁶ pfu/mL. Most preferably, the initial amount of the bacteriophage is between 2.5×10⁶ pfu/mL and 4×10⁶ pfu/mL. Because of the variance in initial levels, a reliable test based on an amplification threshold must use a threshold that is much larger above the mean initial level. Typically, this may be three standard deviations. A threshold-based test thus requires a minimal time of designated T_{T} in FIG. 1 to detect amplification reliably. For the run 24 with the highest initial bacteriophage signal, which in this example has the highest concentration of bacteriophage, the time T_{T1} is the shortest, about 115 minutes. For the run 26 with the second highest signal, which in this case has the second highest initial concentration of bacteriophage, the time T_{T2} is longer, about 145 minutes; and the run 28 with the lowest signal, which in this case has the lowest initial concentration of bacteriophage, the time T_{T3} is about 155 minutes.

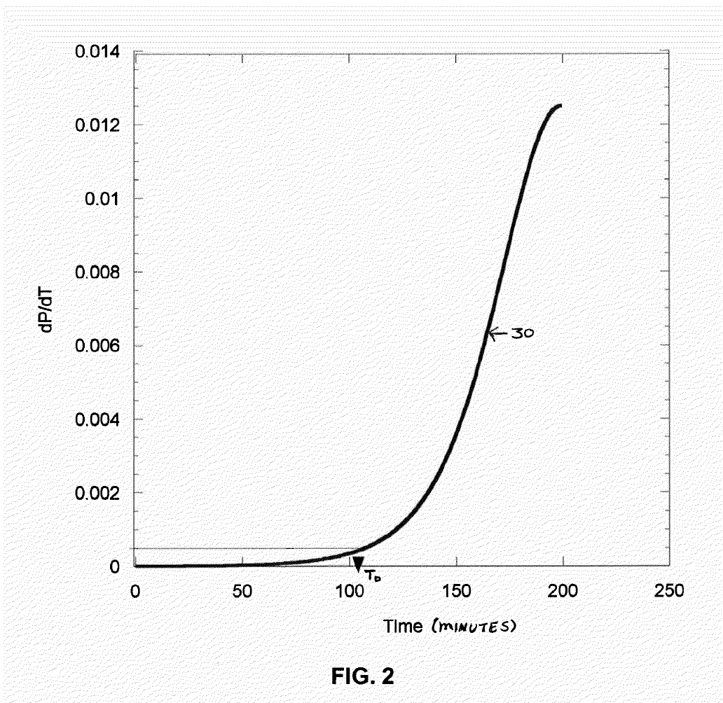

In contrast, an assay that monitors the change in bacteriophage levels over time is insensitive to variations in initial levels. Such an assay that detects a slope of a plot of a bacteriophage marker versus time, the curvature of a plot of the marker versus time, or a change in slope of a plot of the marker versus time, detects bacteriophage amplification more robustly and in less time, as designated T_{D} in FIG. 2, which in this case is about 105 minutes. It is noted that curve 30 is the same for all runs. It is evident that the lower the initial signal, the more the improvement in time to detection. Thus, the method of the invention is particularly useful for low initial signal levels or lower initial concentrations of bacteriophage. Since lower concentrations of bacteriophage can provide better signal to noise, the method of the invention is particularly effective. See United State patent application Ser. No. 12/066,806 filed Mar. 13, 2008, which is hereby incorporated by reference to the same extent as though fully disclosed herein.

The slope of a bacteriophage signal versus time, the curvature of the plot of the bacteriophage signal versus time, or a change in slope of the plot versus time, can be determined in many ways that are known in the art. We refer to the procedure for making one or more of these determinations as an “algorithm” herein. The algorithm may be as simple as simply taking measurements at time intervals and plotting them; or it may be by way of an instrument that detects the change in a bacteriophage measurement. Preferably, the plotting is done electronically. Preferably, the measurement is also taken electronically. For example, a plurality of lateral flow strips as described in United State patent application Ser. No. 12/402,337 filed Mar. 11, 2009 may be used to measure points on the curve. The flow strips may be read with an optical scanner. This patent application is hereby incorporated by reference to the same extent as though fully disclosed herein. A more sophisticated algorithm that can be used with any bacteriophage-based microorganism detection method is disclosed in United State Patent Application Publication No. US2010/0070185 on an invention of Ronald T. Kurnick and Martin Tiz, published on Mar. 18, 2010, which patent application is incorporated by reference to the same extent as though fully disclosed herein.

The bacteria detection processes using bacteriophage can be configured to determine antibiotic susceptibility of the target bacteria; and the invention is also applicable to such an antibiotic susceptibility test. For example, a sample potentially containing target bacteria is divided into two parts: Sample One and Sample Two. A phage amplification process or phage capture assay process measuring the change in bacteriophage concentration or change in the rate of change of the bacteriophage concentration described previously is performed on Sample One to ascertain the presence of the target bacteria in the sample. Samples One and Two are tested simultaneously or serially beginning with Sample One. If the presence of the target bacteria is already known via some other method, then Sample One is not needed nor is the associated phage assay. Sample Two is treated differently. An antibiotic is added to Sample Two at a specific concentration. Then Sample Two is optionally incubated for a predetermined period of time to allow the antibiotic to act upon the target bacteria. A reagent containing phage that is specific to the target bacteria is added to Sample Two; and Sample Two is incubated optionally for a predetermined time. The previously described phage amplification assay process or phage capture binding assay detection process measuring the change in bacteriophage concentration or change in the rate of change of the bacteriophage concentration is performed. If the target bacteria is resistant to the antibiotic, it will grow and a change in bacteriophage concentration or change in the rate of change of bacteriophage concentration is detected in the assay producing a positive result. The positive result indicates that the target bacterium is present in the assay; and the particular strain is resistant to the tested antibiotic. If the target bacterium is susceptible to the tested antibiotic, it will not grow in Sample Two; and the assay result will be negative. This result combined with a positive result on the assay performed on Sample One with no antibiotic will indicate that the target bacteria is present and that it is susceptible to the antibiotic.

Many other phage-based methods and apparatus used to identify the microorganism and/or to determine the antibiotic resistance test or antibiotic susceptibility can be enhanced by the method and apparatus of the invention. For example, a phage amplification process, such as a process described in US Patent Application Publication No. US2005/0003346 entitled “Apparatus And Method For Detecting Microscopic Living Organisms Using Bacteriophage” may be enhanced by the present invention. A process of attaching to a microorganism, such as described in PCT Patent Application Serial No. PCT/US06/12371 entitled “Apparatus And Method For Detecting Microorganisms Using Flagged Bacteriophage” may also be enhanced. Any other phage-based identification process may also be used.

There has been described an improvement to the conventional bacteria detection methods using bacteriophage that overcomes the problem of noise in the measurements. It should be understood that the particular embodiments shown in the drawings and described within this specification are for purposes of example and should not be construed to limit the invention, which will be described in the claims below. Further, it is evident that those skilled in the art may now make numerous uses and modifications of the specific embodiment described, without departing from the inventive concepts. Equivalent structures and processes may be substituted for the various structures and processes described; the subprocesses of the inventive method may, in some instances, be performed in a different order; or a variety of different materials and elements may be used. Consequently, the invention is to be construed as embracing each and every novel feature and novel combination of features present in and/or possessed by the microorganism detection apparatus and methods described.

What is claimed as new and desired to be protected by Letters Patent of the United States is:

1. A method of determining the presence or absence of a target microorganism in a sample to be tested, said method comprising:
(a) combining with said sample an amount of bacteriophage capable of infecting said target microorganism to create a bacteriophage-exposed sample;
(b) providing conditions to said bacteriophage-exposed sample sufficient to allow said bacteriophage to multiply in said target microorganism; and
(c) assaying said bacteriophage-exposed sample to detect the time rate of change of a bacteriophage marker to determine the presence or absence of said target microorganism.

2. A method as in claim 1 wherein said microorganism is a bacterium and said assaying comprises detecting said bacteriophage marker as an indication of the presence of said target bacterium in said sample.

3. A method as in claim 1 wherein said rate of change is the first time derivative or curvature of said bacteriophage marker.

4. A method as in claim 1 wherein said rate of change is the second time derivative of said bacteriophage marker.

5. A method as in claim 1 wherein said assaying comprises applying an algorithm that detects the slope of said marker.

6. A method as in claim 1 wherein the initial amount of bacteriophage comprises a bacteriophage concentration of between 1×10³ pfu/mL and 1×10⁷ pfu/mL.

7. A method of determining the presence or absence of a target microorganism in a sample to be tested, said method comprising:
(a) combining with said sample an amount of bacteriophage capable of infecting said target microorganism to create a bacteriophage-exposed sample;
(b) providing conditions to said bacteriophage-exposed sample sufficient to allow said bacteriophage to multiply in said target microorganism; and
(c) assaying said bacteriophage-exposed sample to detect the time rate of change of the time rate of change in a bacteriophage marker to determine the presence or absence of said target microorganism.

8. A method as in claim 7 wherein said microorganism is a bacterium and said assaying comprises detecting said bacteriophage marker as an indication of the presence of said target bacterium in said sample.

9. A method as in claim 7 wherein said assaying comprises applying an algorithm that detects the change in slope of said marker as a function of time.

10. A method as in claim 7 wherein the initial amount of said bacteriophage comprises a bacteriophage concentration of between 1×10³ pfu/mL and 1×10⁷ pfu/mL.
